(19) United States
(12) Patent Application Publication
(10) Pub. No.: US 2026/0071174 A1
(43) Pub. Date: Mar. 12, 2026

(54) PHOTOCROSSLINKABLE BIOINK COMPOSITION FOR THREE-DIMENSIONAL PRINTING AND METHOD OF FABRICATING BIOCOMPATIBLE THREE-DIMENSIONAL HYDROGEL CONSTRUCT

(71) Applicant: City University of Hong Kong, Hong Kong (HK)

(72) Inventors: Jinlian HU, Hong Kong (HK); Cong WANG, Hong Kong (HK); Qi ZHANG, Hong Kong (HK); Hanbai WU, Hong Kong (HK)

(21) Appl. No.: 19/228,725

(22) Filed: Jun. 4, 2025

Related U.S. Application Data
(60) Provisional application No. 63/655,612, filed on Jun. 4, 2024.

(51) Int. Cl. C12N 5/00; B29C 64/118; B29K 105/00; B29L 31/00; B33Y 10/00; B33Y 40/20; B33Y 70/10; B33Y 80/00; C09D 189/04

(52) U.S. Cl. CPC C12N 5/0062; B29C 64/118; B33Y 10/00; B33Y 40/20; B33Y 70/10; B33Y 80/00; C09D 189/04; B29K 2089/00; B29K 2105/0005; B29K 2105/0058; B29L 2031/753; C12N 2513/00; C12N 2533/50; C12N 2533/54; C12N 2537/10

(57) ABSTRACT

A photocrosslinkable bioink composition for three-dimensional printing is provided. The photocrosslinkable bioink composition includes: an aqueous solution of a recombinant spider silk protein comprising the NT2RepCT-MaSp2 sequence; a polymer comprising pendant groups reactive under visible light-initiated radical polymerization; and a photoinitiator system configured to generate free radicals upon exposure to visible light. The polymer is selected from the group consisting of methacrylated gelatin, methacrylated hyaluronic acid, methacrylated chitosan, polyethylene glycol diacrylate, and combinations thereof. The photoinitiator system is selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate. The photocrosslinkable bioink composition is curable under visible light to form a biocompatible hydrogel structure suitable for cell culture or implantation.

### REFERENCE TO SEQUENCE DISCLOSURE

The Sequence Listing XML file entitled “P3160US01_Sequence Listing.xml”, created on Sep. 9, 2025, having a size of 8,873 bytes, is hereby incorporated by reference in its entirety. The Sequence Listing XML file submitted via Patent Center forms part of the specification of this application.

### TECHNICAL FIELD

The present invention relates to the field of biotechnological three-dimensional (3D) printing, and more particularly, to a photocrosslinkable bioink composition for 3D printing and a method of fabricating biocompatible 3D hydrogel construct.

### BACKGROUND

In the advancement of biotechnological three-dimensional (3D) printing, the development of bio-compatible materials that can be effectively printed under visible light remains a significant challenge. Conventional bio-inks, such as collagen and alginate, are well-regarded for their biocompatibility; however, they exhibit limited performance when crosslinked using visible light. These materials typically require ultraviolet (UV) light or specialized irradiation conditions to achieve effective crosslinking, which may adversely affect cell viability during or after the printing process.

However, UV curing presents several limitations: it has limited penetration depth in biological tissues, generates reactive oxygen species that damage nucleic acids and proteins, and impairs the viability of encapsulated cells. As a result, UV-based systems are suboptimal for applications involving sensitive biological components or live cells.

To overcome these drawbacks, the use of visible light for photopolymerization has emerged as a safer and more biocompatible alternative. Visible light curing offers deeper tissue penetration, reduced cytotoxicity, and compatibility with aqueous and protein-rich formulations. Yet, few bioinks are formulated to polymerize efficiently under visible light, particularly those incorporating structural proteins of biological origin.

Recent developments in gelatin methacryloyl (GelMA)-based hydrogels have attracted attention due to their photocrosslinkable properties and tunable mechanical characteristics. However, even GelMA, despite its widespread use, still exhibits suboptimal performance under visible light irradiation. The need for ultraviolet initiators or prolonged exposure to achieve sufficient crosslinking limits its compatibility with encapsulated living cells and reduces its utility in sensitive bioprinting environments.

Therefore, there is an unmet need for novel bio-ink compositions that offer enhanced printability, structural fidelity, and cytocompatibility under mild visible-light curing conditions.

### SUMMARY OF INVENTION

It is an objective of the present invention to provide a method to address the aforementioned shortcomings and unmet needs in the state of the art.

In accordance with a first aspect of the present invention, a photocrosslinkable bioink composition for three-dimensional printing is provided.

The photocrosslinkable bioink composition includes: an aqueous solution of a recombinant spider silk protein comprising the NT2RepCT-MaSp2 sequence; a polymer comprising pendant groups reactive under visible light-initiated radical polymerization; and a photoinitiator system configured to generate free radicals upon exposure to visible light. The polymer is selected from the group consisting of methacrylated gelatin, methacrylated hyaluronic acid, methacrylated chitosan, polyethylene glycol diacrylate, and combinations thereof. The photoinitiator system is selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate. The photocrosslinkable bioink composition is curable under visible light to form a biocompatible hydrogel structure suitable for cell culture or implantation.

In accordance with a second aspect of the present invention, a method of fabricating a biocompatible three-dimensional hydrogel construct is provided. The method includes: providing a bioink composition comprising a recombinant spider silk protein comprising the NT2RepCT-MaSp2 sequence; a visible light-reactive polymer selected from the group consisting of methacrylated gelatin, methacrylated hyaluronic acid, methacrylated chitosan, polyethylene glycol diacrylate, and combinations thereof; and a photoinitiator selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate. The method further includes: dispensing the bioink composition into a defined three-dimensional pattern using a printing device; and exposing the patterned bioink to visible light in the range of 405 to 520 nanometers to induce photocrosslinking and form a structurally stable hydrogel, wherein a resulting printed construct is configured to support cell adhesion, proliferation, or tissue integration.

In accordance with a third aspect, a three-dimensional (3D) printing bioink composition is provided. The 3D printing bioink composition includes: a spider silk protein; a photocrosslinkable polymer comprising a pendant group reactive under visible light; a visible light photoinitiator; and a biocompatible aqueous carrier.

In accordance with a fourth aspect, a method of fabricating a biocompatible 3D structure, comprising steps of: providing the 3D printing bioink composition as afore-mentioned; extruding the composition into a defined geometry; and exposing the extruded material to visible light to form a crosslinked hydrogel structure.

### BRIEF DESCRIPTION OF DRAWINGS

Embodiments of the invention are described in more details hereinafter with reference to the drawings, in which:

FIG. 1 illustrates a schematic flowchart for preparing a bio-ink composition suitable for printing in three-dimensional (3D) bioprinting according to some embodiments of the present invention;

FIG. 2 illustrates the assembled NT2RepCT-MaSp2 gene sequence used in this example;

FIG. 3 provides the sequencing chromatograms of both the wild-type and mutated NT2RepCT-MaSp2 sequences;

FIG. 4 shows the expression and purification profile of the TrxA-6×His-HRV3C site-tagged NT2RepCT recombinant spider silk protein in E;

FIG. 5 illustrates the rheological properties of the bio-ink formulated in Example 4;

FIG. 6 shows a schematic representation of the printing process described in Example 4 according to some embodiments of the present invention;

FIG. 7 illustrates the temperature-dependent printability of the bio-ink described in Example 4;

FIG. 8 illustrates the influence of printing parameters on scaffold formation, as applied in Example 4;

FIG. 9 shows fluorescence images of the biocompatibility of the printed scaffolds described in Example 4;

FIG. 10 presents the quantitative evaluation of cytotoxicity and cell proliferation for the scaffolds printed in Example 4; and

FIG. 11 shows confocal fluorescence microscopy images of the cell-laden scaffold printed in Example 5.

### DETAILED DESCRIPTION OF THE INVENTION

In the following description, photocrosslinkable bioink compositions for 3D printing and methods of fabricating biocompatible 3D hydrogel constructs and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

### I. Overview of 3D Printable Biocompatible Inks

The present invention provides 3D printable biocompatible inks that are curable using visible like and serve as bio-inks with a photocrosslinkable bioink composition. The inks include a protein component, such as a recombinant spider silk protein. The ink further includes a visible light photocrosslinkable polymer comprising at least one pendant group reactive under visible light irradiation. A visible light photoinitiator system is used to initiate crosslinking of the visible light crosslinkable polymer. Additional ingredients may optionally be included such as an aqueous buffer suitable for 3D printing applications.

The composition is formulated to remain in a printable liquid or gel state until exposure to visible light (e.g., blue or green wavelengths), whereupon it undergoes crosslinking to form a solid or semi-solid hydrogel network. The system is biocompatible and may be used in the fabrication of structures including, such as tissue engineering scaffolds, cell-laden hydrogels for in vitro modeling, implantable biostructures, wound healing matrices, drug delivery platforms, and organoid or tumor models.

The protein component may be a spider silk protein. Spider silk proteins are an attractive material for use in bioprinting due to their unique mechanical and biological properties. Spider silk proteins (e.g., scientifically known as spidroins) are the structural components of natural spider silk fibers. Spiders produce multiple types of silk for different functions (e.g., dragline, capture spiral), but the most mechanically impressive is dragline silk, composed primarily of Major Ampullate Spidroin 1 (MaSp1) and Major Ampullate Spidroin 2 (MaSp2). In general, MaSp1 has higher tensile strength while MaSp2 has higher elasticity.

Recombinant silk variants, such as the NT2RepCT-MaSp2 construct, derived from major ampullate spidroin-2, offer high extensibility, thermal stability, and biodegradability. This protein includes the N-terminal domain (NT), two tandem repeats (2Rep), a central repetitive sequence modeled on MaSp2, and a C-terminal domain (CT). The sequence design facilitates solubility, self-assembly, and mechanical integrity. The protein may be expressed in microbial systems and purified using standard techniques Such silk proteins are known to exhibit low immunogenicity and can be engineered with cell-binding motifs such as RGD sequences, promoting cell adhesion and tissue integration. These features make spider silk an exceptional matrix for scaffolds intended for cell culture, wound healing, or implantation.

However, other spider silk proteins may also be used based on the particular mechanical properties of the printed object. For example, particular spider silk proteins may be selected based on whether high tensile strength or high elasticity is needed in the final product. Further, other bio-proteins such as silk fibroin from silkworms, elastin-like polypeptides (ELPs), mussel foot proteins, or recombinant collagen-like proteins may also be used in place of or in addition to the spider silk protein.

The visible light curable polymer component of the bioink includes functional groups suitable for radical polymerization. Preferred materials include methacrylated gelatin (GelMA), methacrylated hyaluronic acid (HAMA), methacrylated chitosan (ChiMA), methacrylated alginate, norbornene-functionalized polyethylene glycol (PEGNB), and polyethylene glycol diacrylate (PEGDA). These polymers form hydrogels with tunable stiffness, degradation rates, and pore architectures. Mixtures of two or more polymers may be used to optimize printing and biological performance. In one embodiment, the polymer comprises methacrylated gelatin and the resulting cured hydrogel exhibits an elastic modulus between 5 and 50 kilopascals, suitable for soft tissue applications.

The visible light photoinitiator system comprises compounds capable of generating radicals under visible light. Exemplary systems include tris(bipyridine)ruthenium(II) chloride combined with ammonium persulfate (Ru(bpy)_3²⁺/APS), which activates under blue-green light (˜450-520 nm). Other examples include eosin Y with triethanolamine, riboflavin with ammonium persulfate, camphorquinone with an amine co-initiator, and lithium phenyl-2,4,6-trimethylbenzoylphosphinate (LAP). The concentration of the photoinitiator is selected to balance curing speed, depth, and cytocompatibility.

Importantly, because the biocompatible inks are cured using visible light, various cells may be incorporated into the ink along with cell support components such as growth media and growth factors. Optional components such as growth factors, peptide motifs, or encapsulated cells may be included.

The bioink is loaded into a 3D printing system and extruded or digitally patterned into a predesigned shape. The printed construct is then irradiated with visible light to initiate crosslinking. The resulting hydrogel may serve various purposes:

Wound healing scaffold: Promotes epithelial or stromal cell growth in situ.

Tissue engineering lattice: Supports fibroblast, chondrocyte, or stem cell integration.

In vitro cell culture matrix: Enables long-term cell culture in a tunable 3D environment.

Tumor or organoid model: Provides physiologically relevant conditions for drug testing.

Implantable hydrogel film or disk: Offers load-bearing or bioactive interface with host tissues.

In one embodiment, the printed construct is configured as a wound healing scaffold comprising a porous hydrogel network infused with the spider silk protein, and wherein the scaffold supports epithelial cell migration across the wound bed. In one embodiment, the printed construct comprises a tissue engineering scaffold having a three-dimensional lattice structure configured to promote attachment and proliferation of fibroblasts, chondrocytes, or stem cells.

### II. Formation of Particular Bioink Compositions

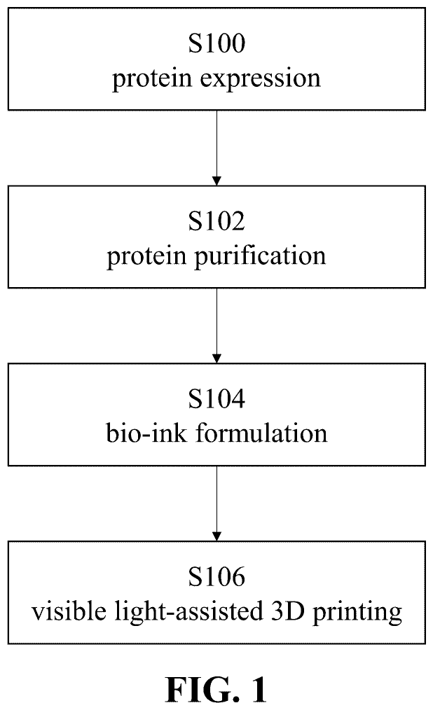

FIG. 1 illustrates a schematic flowchart for preparing a bio-ink composition suitable for printing in three-dimensional (3D) bioprinting according to some embodiments of the present invention. The disclosed method also represents the general production route for the bio-ink and its subsequent use in fabricating a cell-compatible 3D scaffold. The method includes the following steps: protein expression (step S100), protein purification (S102), bio-ink formulation (step S104), and visible light-assisted 3D printing (step S106).

### Step S100: Expression of Recombinant Spider Silk Protein

In this step, a positive colony of Escherichia coli is selected for recombinant protein expression and initially cultured in Lysogeny Broth (LB) medium containing ampicillin. The bacterial suspension is incubated in a shaking incubator for 12 hours to allow for initial cell growth. After this stage, the culture is transferred to 1 liter of LB medium supplemented with kanamycin to establish a high-density fermentation environment. Cultivation is continued under shaking at 225 revolutions per minute (rpm) at a temperature of 37° C. until the optical density at 600 nanometers (OD₆₀₀) reaches a value between 0.8 and 1.0. At this point, isopropyl β-D-1-thiogalactopyranoside (IPTG) is added to induce protein expression. Simultaneously, the incubation temperature is reduced to 15° C., and the culture is maintained for an additional 18 to 20 hours to minimize the formation of inclusion bodies and to increase the final protein yield.

In some embodiments, the expression host is E. coli BL21 (DE3), and the LB medium contains 100 μg/mL ampicillin in the initial stage and 100 μg/mL kanamycin in the subsequent high-density culture stage. The shaking incubator operates at 225 rpm and 37° C. IPTG is added to the culture at a final concentration of 0.25 mM to induce recombinant protein expression.

The bacterial culture obtained in Step S100, which contains the expressed protein but has not undergone purification, is herein referred to as the “unpurified recombinant spider silk protein solution.”

### Step S102: Protein Purification

In this step, the unpurified recombinant spider silk protein solution obtained from Step S100 is subjected to purification. Following extended cultivation, the bacterial cells are harvested by centrifugation at 5000 revolutions per minute (rpm) for 30 minutes at 4° C. The resulting cell pellet is resuspended in lysis buffer at a ratio of 1:4 (w/v) and stored at −80° C. overnight to facilitate cell disruption. The resuspended cells are then lysed using a high-pressure homogenizer operating at 800 bar for 20 minutes at 4° C. to ensure complete cellular breakdown. The lysate is subsequently centrifuged at 20,000 rpm for 1 hour at 4° C.

The supernatant is collected for downstream protein purification, while the pellet is retained at −20° C. for SDS-PAGE analysis. The purification of the recombinant spider silk protein is initiated under native conditions using immobilized metal affinity chromatography (IMAC) on an ÄKTA Pure™ system. The supernatant is passed through a 5 mL Ni-NTA column. Impurity proteins are removed using a washing buffer, and the target protein is eluted using an elution buffer. The eluted fractions containing the target protein are incubated with HRV 3C protease at 4° C. for 12 hours to cleave the 6×-His and Trx tags. The cleaved protein is subsequently subjected to size-exclusion chromatography using a GB buffer to achieve final purification. Protein purity is verified via SDS-PAGE analysis. The resulting protein solution is then concentrated using a centrifugal ultrafiltration device with a molecular weight cut-off (MWCO) of 10 kDa. The concentrate is freeze-dried and stored at 4° C. in powder form for subsequent use.

In some embodiments, the purification process employs the following buffers: the lysis buffer comprises 20 mM Tris-HCl (pH 8.0), 1 mM PMSF, and 0.25% Tween-20; the impurity washing buffer comprises 20 mM Tris-HCl (pH 8.0), 5 mM imidazole, and 0.05% Tween-20; the elution buffer comprises 20 mM Tris-Cl (pH 8.0), 250 mM imidazole, and 0.05% Tween-20; and the GB buffer used for size-exclusion chromatography comprises 20 mM Tris-Cl (pH 8.0), 1 mM EDTA, and 0.05% Tween-20.

The final powdered product obtained in Step S102 is hereinafter referred to as the “recombinant spider silk protein powder.”

That is, the production process begins with the use of genetically engineered E. coli cells harboring a recombinant plasmid encoding the spider silk protein of interest. These host cells are cultured under standard conditions to promote biomass accumulation and recombinant protein expression. Upon reaching the desired expression phase, the resulting bacterial culture, which contains intracellularly accumulated protein, is herein referred to as the unpurified recombinant spider silk protein solution. This intermediate material comprises both the expressed protein and cellular components within an intact or partially lysed matrix. Following expression, the culture is subjected to a sequence of operations including cell harvesting, mechanical disruption, clarification, and multi-step chromatographic purification. Through this transformation, non-target substances are removed, and the recombinant spider silk protein is progressively enriched. Upon completion of tag removal and protein concentration, the final product is obtained as a stable, freeze-dried powder suitable for storage and subsequent formulation. The resulting purified protein material at the end of this stage is referred to as the recombinant spider silk protein powder, which serves as a functional intermediate for subsequent bio-ink formulation. In some embodiment, the recombinant spider silk protein comprises one or more terminal groups functionalized with methacrylate or acrylate moieties to facilitate covalent incorporation into the photocrosslinked network.

### Step S104: Preparation of the 3D Bioprinting Ink

In this step, a 3D bioprinting ink is prepared by dissolving 1 gram of methacrylated gelatin in 10 milliliters of deionized water. The solution is heated to 50° C. and stirred until fully dissolved. After cooling to room temperature, 0.1 gram of the recombinant spider silk protein powder obtained from Step S102 is added to the solution. In addition, 0.1 gram of tris(bipyridine)ruthenium(II) chloride solution and 0.1 gram of ammonium persulfate solution are introduced. In some embodiment, the recombinant spider silk protein powder obtained from Step S102 is added to the solution at a concentration ranging from 1 weight percent to 3 weight percent of the total formulation. The resulting mixture is stirred thoroughly under ambient temperature in a light-protected environment until homogenous. The completed bio-ink formulation is then transferred into a black opaque syringe, where air bubbles are removed. The filled syringe is stored at 4° C. in the dark until further use.

In some embodiments, the methacrylated gelatin used in the formulation has a degree of methacrylation of approximately 60% and a gel strength of about 250 gram-force (G). The tris(bipyridine)ruthenium(II) chloride solution has a concentration of approximately 15 mg/mL, and the ammonium persulfate solution also has a concentration of approximately 15 mg/mL.

In one embodiment, the tris(bipyridine)ruthenium(II) chloride serves as the photoinitiator, and ammonium persulfate acts as the co-initiator or electron donor. Together, they constitute a visible light-responsive photoinitiator. In other embodiments, the concentrations of the photoinitiator and co-initiator may be varied according to performance requirements. While the above procedure achieves a 10 wt % concentration of methacrylated gelatin based on the use of 1 gram in 10 milliliters of water, the present invention is not limited to this specific formulation.

### Step S106: Printing of the 3D Bioprinting Ink

In this step, the bio-ink prepared in Step S104 is retrieved and loaded into a 3D bioprinter with an ink cartridge which is connected to a pneumatic extrusion system. In some embodiments, the bio-ink with a photocrosslinkable bioink composition comprising one or more fibroblasts which are suspended in the aqueous phase, and the photocrosslinking conditions are cytocompatible such that cell viability of the one or more fibroblasts is retained following visible light exposure. During the printing process, the bio-ink is extruded through a dispensing nozzle while simultaneously being irradiated with a 432 nm blue light source (e.g., the visible light exposure is provided by a digital light projector or laser emitting light at approximately 432 nanometers), thereby initiating photocrosslinking and forming a structurally defined three-dimensional scaffold (e.g., hydrogel structure). The 432 nm blue light may be provided by a visible light LED source, such as a high-intensity collimated LED array or a laser diode system equipped with optical focusing elements. The 432 nm blue light, in conjunction with the tris(bipyridine)ruthenium(II) chloride solution and ammonium persulfate solution included in the bio-ink, collectively function as a photoinitiating system capable of inducing visible light-triggered crosslinking during the printing operation.

In some embodiments, during the extrusion-based printing process, the printing device temperature (e.g., syringe temperature) is maintained between approximately 22.5° C. and 25° C. The printing platform may be cooled to approximately 15° C., for example, by using a thermoelectric cooling module or a circulating refrigerated stage. The pneumatic extrusion pressure may range from 0.15 MPa to 0.23 MPa. The printing speed may be within the range of 20 mm/s to 40 mm/s. The nozzle gauge may be selected from among 23G to 27G, depending on the desired resolution and flow characteristics of the ink.

By the configuration above, as compared with other manners in the prior art, the present invention provides the following advantages, each of which is attributable to specific structural features and processing techniques described in Steps S100 through S106:
(1) Improved Printability: By incorporating a combination of methacrylated gelatin (GelMA) and recombinant spider silk protein, the resulting bio-ink demonstrates superior printability under visible light exposure. The GelMA component, characterized by its methacrylate substitution and tunable gel strength, imparts rapid photo-crosslinking capability, while the spider silk protein, produced and purified through a multistep process (Steps S100-S102), enhances the mechanical stability and shear-thinning behavior of the ink. Together, these components yield a rheologically optimized system that supports precise layer-by-layer deposition and maintains structural fidelity during extrusion, enabling the fabrication of complex and finely resolved biological constructs.
(2) Enhanced Biocompatibility: The use of recombinant spider silk protein, expressed in E. coli and subsequently purified to remove host contaminants and tags (Steps S100-S102), provides a bio-derived matrix component known for its inherent cytocompatibility. Unlike synthetic polymers or UV-curable monomers, spider silk proteins support cell adhesion and proliferation, contributing to a biologically favorable environment when embedded in hydrogel scaffolds. This makes the resulting ink particularly suitable for biomedical applications such as tissue engineering and regenerative medicine.
(3) Mild Crosslinking Conditions: The photocuring strategy adopted in this invention utilizes a visible light photoinitiating system, comprising tris(bipyridine)ruthenium(II) chloride and ammonium persulfate (introduced in Step S104), which is activated by a 432 nm blue light source (Step S106). This replaces conventional ultraviolet-based systems that often compromise cell viability due to higher energy exposure. The visible light regime enables rapid gelation at ambient temperatures while preserving cellular functionality, which is critical for cell-laden bio-inks and post-printing cell survival within the scaffold.
(4) Environmentally Friendly Formulation: The bio-ink developed herein does not rely on toxic photoinitiators, organic solvents, or harsh crosslinking agents. All components (i.e., including deionized water as the solvent, recombinant proteins from microbial expression, and aqueous-based initiator systems) are selected for their low toxicity and biodegradability. As a result, the formulation is safer for laboratory handling and more compatible with clinical and in vivo use, aligning with growing demand for sustainable biomanufacturing.
(5) Broad Application Potential: Due to its combined advantages in printability, biocompatibility, and mild curing, the bio-ink described herein can be applied to a wide variety of 3D bioprinting scenarios. These include but are not limited to the fabrication of structurally defined tissue scaffolds, disease models for pathophysiological research, and high-throughput platforms for drug screening. The tunability of material composition and crosslinking conditions further extends its versatility across different cell types and printing systems.

The following description provides a more detailed explanation of Steps S100 through S106, including representative materials, process conditions, and variations that may be adopted in different embodiments. Each step is discussed in relation to its functional role in the overall process of preparing and applying the bio-ink formulation.

### EXAMPLES

### Example 1: Construction and Site-Directed Mutagenesis of the Recombinant NT2RepCT-MaSp2 Gene

This example corresponds to the upstream genetic construction aspect of Step S100, specifically detailing the synthesis and site-directed mutagenesis of the recombinant spider silk protein gene.

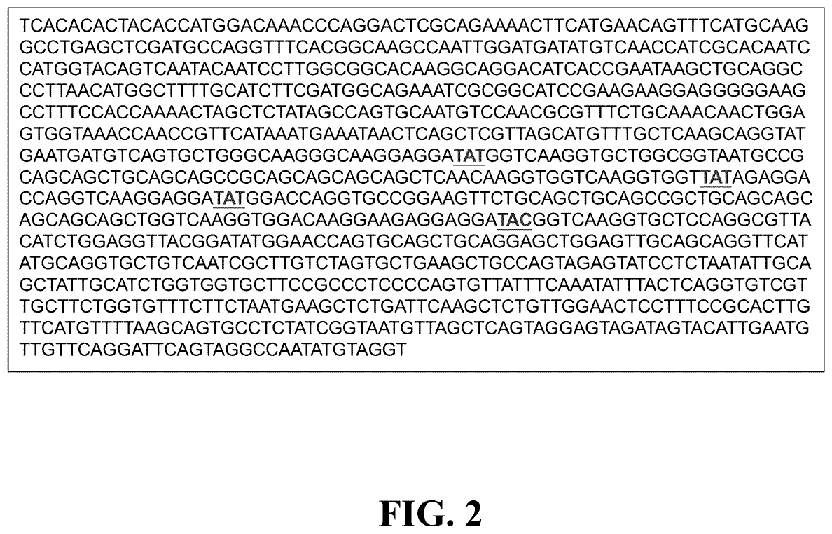

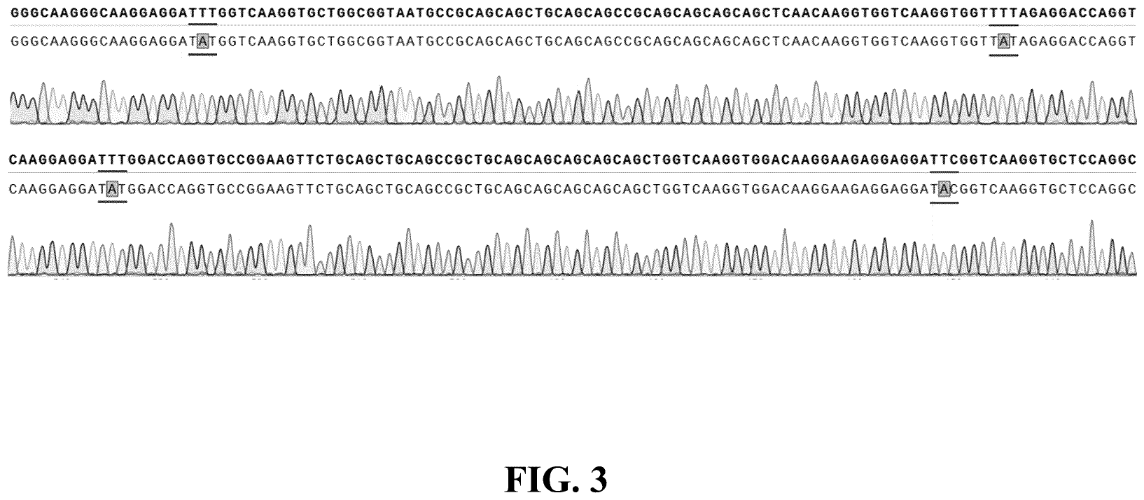

FIG. 2 illustrates the assembled NT2RepCT-MaSp2 gene sequence used in this example. The mutation sites introduced during PCR mutagenesis are indicated by underlining. FIG. 3 provides the sequencing chromatograms of both the wild-type and mutated NT2RepCT-MaSp2 sequences. The top panel corresponds to the original sequence, while the bottom panel shows the modified sequence. Underlines denote the confirmed mutation positions.

In this example, a recombinant spider silk protein gene encoding NT2RepCT-MaSp2 is constructed and subjected to site-directed mutagenesis. The gene sequence is designed by combining three specific domains: the N-terminal domain of E. australis MaSp1 (EMBL accession no. AM259067), the C-terminal domain of A. ventricosus MiSp (GenBank accession no. JX513956), and the repetitive domain of E. australis MaSp2 (GenBank accession no. AM490192). The full-length synthetic DNA is commercially synthesized by GenScript Biotech.

To introduce targeted point mutations, the synthetic DNA template is amplified using PCR with the primers listed in Table 1. The resulting linear fragments containing the desired mutations are circularized by homologous recombination, and the final constructs are sequence-verified to confirm the successful incorporation of each mutation. The verified mutant sequence of NT2RepCT-MaSp2 is subsequently cloned into a modified pET-32M.3C expression vector, which has been previously engineered in the laboratory to enhance recombinant protein yield.

TABLE 1

| Primer | |
|---|---|
| Name | Primer Sequence (5′-3′) |
| F140Y | AGGAGGATATGGTCAAGGTGCTGGCGGTAATG |
| Forward | |
| (SEQ ID | |
| NO: 01) | |
| F140Y | CTTGACCATATCCTCCTTGCCCTTGCCCAGCA |
| Reverse | |
| (SEQ ID | |
| NO: 02) | |
| F167/ | TAGAGGACCAGGTCAAGGAGGATATGGACCAG |
| 175Y | GTGCCGGAAG |
| Forward | |
| (SEQ ID | |
| NO: 03) | |
| F167/ | CTTGACCTGGTCCTCTATAACCACCTTGACCA |
| 175Y | CCTTGTTG |
| Reverse | |
| (SEQ ID | |
| NO: 04) | |
| F203Y | AGGAGGATACGGTCAAGGTGCTCCAGGCGTTA |
| Forward | |
| (SEQ ID | |
| NO: 05) | |
| F203Y | CTTGACCGTATCCTCCTCTTCCTTGTCCACCT |
| Reverse | TGAC |
| (SEQ ID | |
| NO: 06) | |

### Example 2: Expression of Recombinant Spider Silk Protein

This example corresponds to Step S100, and describes in detail the procedure for expressing the recombinant spider silk protein using a bacterial host.

A positive colony of Escherichia coli BL21 (DE3) is selected for use as the expression strain. The colony is inoculated into 10 mL of Lysogeny Broth (LB) medium containing 100 μg/mL ampicillin and incubated in a shaking incubator at 225 rpm and 37° C. for 12 hours. After this initial culture stage, the bacterial suspension is transferred into 1 liter of LB medium supplemented with 100 μg/mL kanamycin to initiate high-density cultivation.

Cultivation is continued under the same temperature and agitation conditions until the optical density at 600 nm (OD₆₀₀) reaches a value in the range of 0.8 to 1.0, which generally corresponds to the exponential phase of bacterial growth. This stage provides metabolically active cells with a high capacity for recombinant protein expression, making it suitable for the subsequent induction step. At that point, isopropyl β-D-1-thiogalactopyranoside (IPTG) is added to a final concentration of 0.25 mM to induce recombinant protein expression. The incubation temperature is subsequently reduced to 15° C. to suppress inclusion body formation, and the culture is maintained for an additional 18 to 20 hours to promote protein solubility and increase the overall yield.

### Example 3: Purification of Recombinant Spider Silk Protein

This example corresponds to Step S102 and provides the detailed procedure for purifying the recombinant spider silk protein obtained from the expression step.

Following extended cultivation, bacterial cells are harvested by centrifugation at 5000 rpm for 30 minutes at 4° C. The resulting cell pellet is resuspended in lysis buffer at a 1:4 (w/v) ratio. The lysis buffer comprises 20 mM Tris-HCl (pH 8.0), 1 mM PMSF, and 0.25% Tween-20. The suspension is stored overnight at −80° C. to facilitate cell disruption. Subsequently, the suspension is subjected to high-pressure homogenization at 800 bar for 20 minutes at 4° C. to ensure complete cell lysis. In the context of the present disclosure, “complete cell lysis” refers to a disruption state in which intracellular proteins are effectively released into the supernatant, minimizing the presence of intact cells and maximizing the availability of soluble target protein for purification. This condition is characterized by the absence of intact cellular morphology under microscopic observation and a significant reduction in pellet density upon high-speed centrifugation.

The lysate is then centrifuged at 20,000 rpm for 1 hour at 4° C., and the supernatant is collected for purification. Initial purification is conducted under native conditions using immobilized metal affinity chromatography (IMAC) on an ÄKTA Pure™ system (GE Healthcare, Uppsala, Sweden). The supernatant is loaded onto a 5 mL Ni-NTA column (Ni NTA-HisTalon μSphere, PAE, Wuxi, China). Impurity proteins are removed using a wash buffer containing 20 mM Tris-HCl (pH 8.0), 5 mM imidazole, and 0.05% Tween-20.

The target protein is eluted with an elution buffer composed of 20 mM Tris-Cl (pH 8.0), 250 mM imidazole, and 0.05% Tween-20. The collected fractions containing the target protein are incubated at 4° C. for 12 hours with HRV 3C protease to cleave off the 6×-His and Trx fusion tags. The cleaved protein is further purified using size exclusion chromatography on a HiLoad 26/600 Superdex 200 pg column (GE Healthcare, Uppsala, Sweden) with a GB buffer composed of 20 mM Tris-Cl (pH 8.0), 1 mM EDTA, and 0.05% Tween-20.

The purity of the eluted fractions is confirmed by SDS-PAGE. The final protein solution is concentrated using 10 kDa MWCO centrifugal ultrafiltration tubes (Merck Millipore, Germany), and the resulting concentrate is freeze-dried using a Heto Powerdry LL3000 system (ThermoFisher, Massachusetts, USA) to obtain a stable recombinant spider silk protein powder suitable for storage and downstream use.

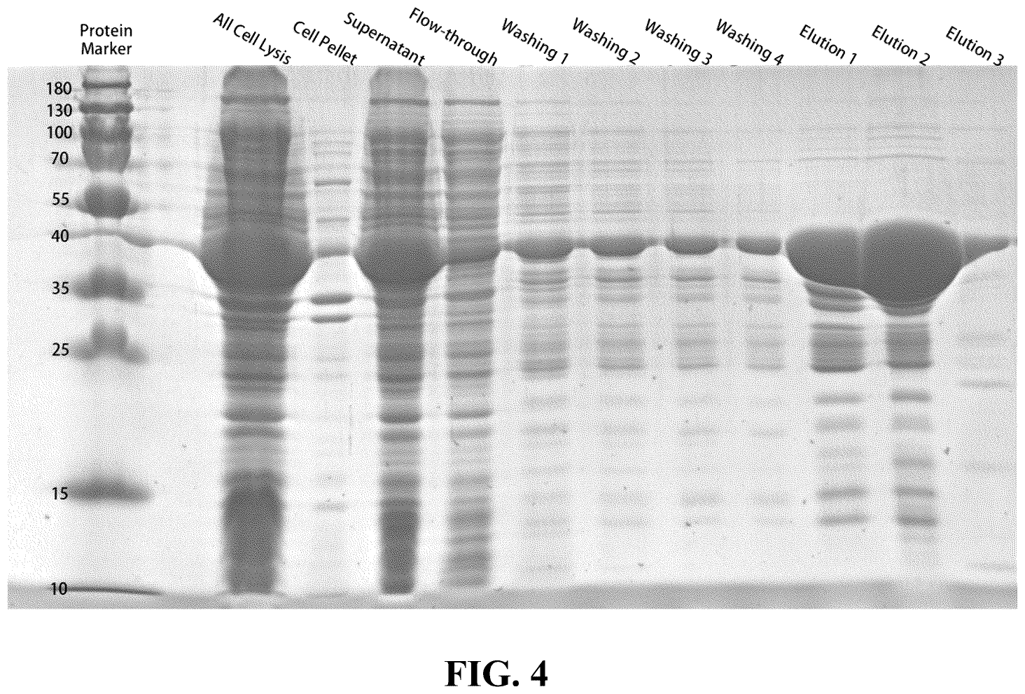

FIG. 4 shows the expression and purification profile of the TrxA-6×His-HRV3C site-tagged NT2RepCT recombinant spider silk protein in E. coli BL21 (DE3), as analyzed by SDS-PAGE. The lane labeled “All Cell Lysis” represents the total protein content following induction and lysis. “Cell Pellet” and “Supernatant” correspond to the insoluble and soluble fractions after centrifugation, respectively. “Flow-through” and “Washing 1-4” show fractions collected during the Ni-NTA affinity chromatography purification step, while “Elution 1-3” represent the purified protein eluted from the column. The illustration in FIG. 4 confirms the enrichment of the target protein in the elution fractions, consistent with the purification procedure described in Example 3.

### Example 4: Fabrication of a 3D-Printed Hydrogel Scaffold

This example corresponds to Steps S104 and S106 and describes the preparation of a bio-ink containing recombinant spider silk protein and the subsequent printing of a three-dimensional hydrogel scaffold under visible light irradiation.
1. Preparation of the 3D Bioprinting Ink: One gram of methacrylated gelatin (degree of substitution: 60%) is added to 10 mL of deionized water and stirred at 50° C. until fully dissolved. After cooling to room temperature, 0.1 g of recombinant spider silk protein powder obtained in Example 2 is added and mixed thoroughly to form Solution A. Separately, 60 mg of tris(bipyridine)ruthenium(II) chloride is dissolved in 4 mL of deionized water, and 120 mg of ammonium persulfate is dissolved in another 4 mL of deionized water. Both are stored at 2-8° C. in the dark. A volume of 0.5 mL from each initiator solution is added to Solution A, and the resulting mixture is transferred into a 30 mL black, opaque cartridge. Air bubbles are removed under vacuum, and the ink is stored at 2-8° C. in the dark until use.

2. 3D Printing of the Hydrogel Scaffold: The bio-ink is loaded into the print head of a regenovo bioprinter. A 432 nm blue light source is directed onto the printing platform, which is maintained at 10° C. The ink cartridge is held at 25° C. and allowed to equilibrate for 10-20 minutes. Printing is initiated under simultaneous extrusion and visible light irradiation. The nozzle gauge is 27G, the print speed is 20 mm/s, and the extrusion pressure is 0.2 MPa. After the scaffold is printed, additional blue light exposure is applied for 1 minute to ensure complete crosslinking. The resulting scaffold has a length and width of 15 mm, a height of 6 mm, and a grid spacing of 0.54 mm.

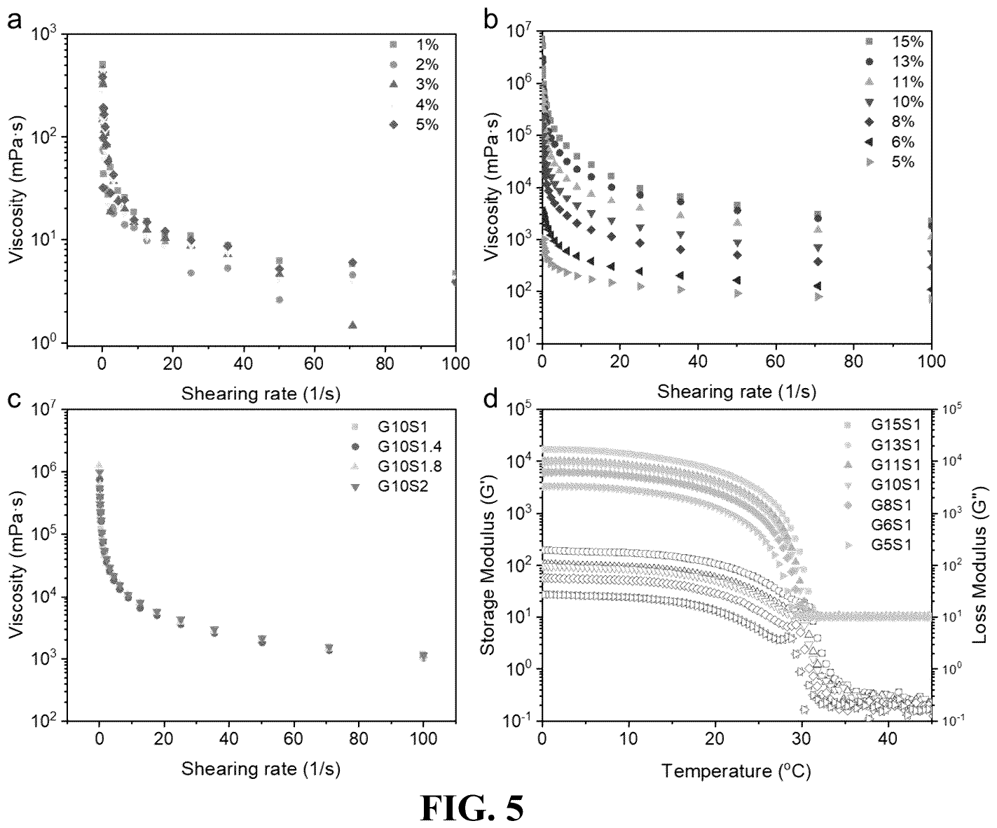

FIG. 5 illustrates the rheological properties of the bio-ink formulated in Example 4, confirming its non-Newtonian behavior, shear-thinning characteristics, and temperature-dependent viscoelasticity, all of which contribute to its suitability for extrusion-based 3D bioprinting. There four parts in the illustration of FIG. 5, including part (a), part (b), part (c), and part (d).

Part (a) of FIG. 5 shows the viscosity profiles of gelatin-only formulations with varying concentrations (1%-5%). All samples exhibit shear-thinning behavior, where viscosity decreases with increasing shear rate, indicating flowability under extrusion conditions. Part (b) of FIG. 5 shows viscosity profiles for gelatin-spider silk composite inks with higher gelatin concentrations (5%-15%). As gelatin content increases, viscosity rises markedly, but shear-thinning remains evident across all formulations, supporting tunability for printability control. Part (c) of FIG. 5 compares the effect of varying spider silk content at constant gelatin concentration. The spider silk additions cause a moderate increase in baseline viscosity, while preserving favorable shear-thinning flow behavior across tested shear rates. Part (d) of FIG. 5 shows the viscoelastic modulus as a function of temperature. The storage modulus (G′) and loss modulus (G″) profiles show a sharp drop above 30° C., indicating thermal softening behavior. This temperature sensitivity allows precise thermal control during extrusion, ensuring print fidelity at room temperature and fluidization at elevated temperatures.

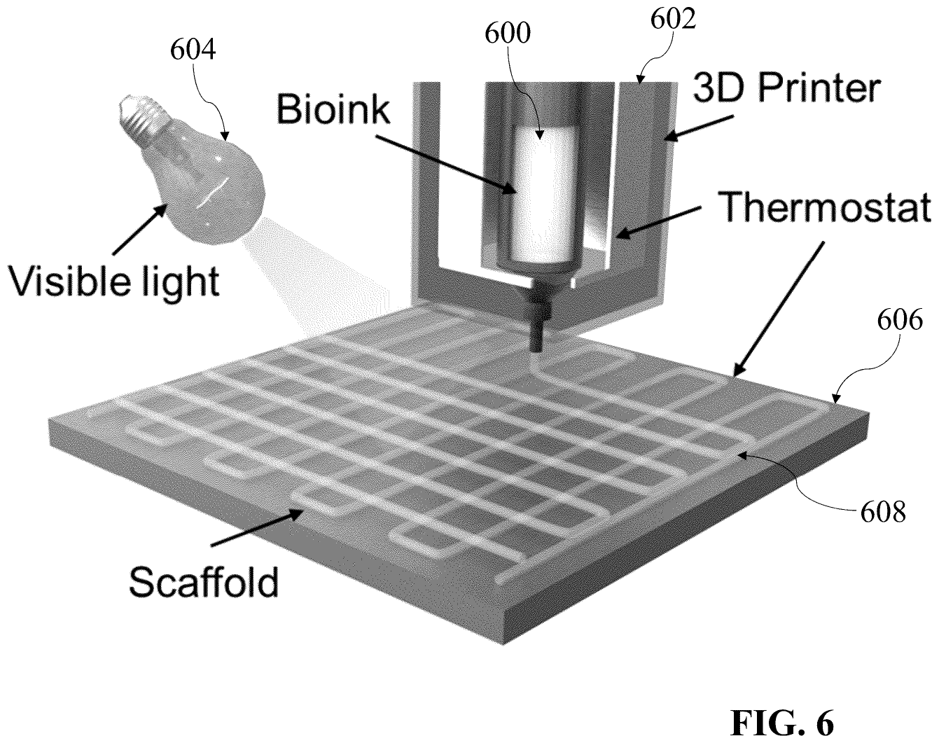

FIG. 6 shows a schematic representation of the printing process described in Example 4 according to some embodiments of the present invention. The bio-ink 600, formulated with methacrylated gelatin and recombinant spider silk protein, is loaded into a temperature-controlled cartridge within a 3D bioprinter (i.e., 3D printer 602). As the bio-ink 600 is extruded through the nozzle, it is simultaneously exposed to visible light provided by a visible light source 604, specifically at a wavelength of 432 nm, which activates the photoinitiator embedded in the ink formulation. The schematic illustrates the coordination between thermal regulation and photopolymerization. The thermostat 606 maintains the bio-ink 600 at an optimal temperature to preserve its viscoelastic properties, while the visible light source 604 initiates rapid crosslinking at the point of deposition. As a result, the extruded filament solidifies into a defined scaffold geometry layer by layer, achieving high shape fidelity and structural integrity. This configuration demonstrates how the integration of visible-light curing and real-time extrusion enables the fabrication of stable, biocompatible hydrogel scaffolds 610 under mild conditions suitable for cell-laden printing applications.

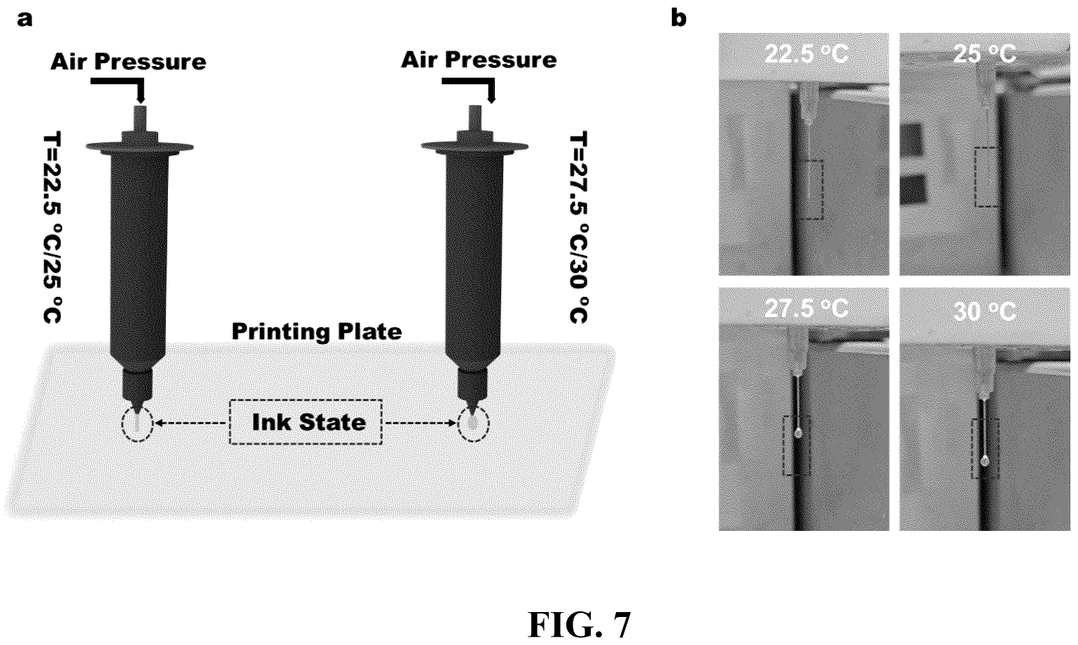

FIG. 7 illustrates the temperature-dependent printability of the bio-ink described in Example 4. The left portion, part (a) of FIG. 7, schematically represents the relationship between cartridge temperature and ink state during extrusion. The right panel, part (b) of FIG. 7, presents experimental observations of extrusion behavior at four controlled temperatures: 22.5° C., 25° C., 27.5° C., and 30° C.

At 22.5° C. and 25° C., the bio-ink maintains a semi-solid, viscoelastic state with sufficient yield stress to support filament formation. As shown in the upper two images of part (b), the ink is smoothly extruded in continuous lines without droplet formation. This behavior reflects an optimal viscosity window for extrusion-based printing, in which the ink flows under applied shear but retains shape after deposition (i.e., printable viscosity).

In contrast, at elevated temperatures of 27.5° C. and 30° C., the ink exhibits a significant loss in viscosity and yield strength, resulting in uncontrolled droplet formation instead of filament extrusion. As shown in the lower two images of part (b), the ink is extruded as spherical droplets rather than linear structures. This thermal transition highlights the ink's sensitivity to temperature, and further supports the use of precise thermal regulation (e.g., thermostated printhead and platform) during the printing process to ensure print fidelity.

The illustration emphasizes the necessity of maintaining the bio-ink within a defined thermal range to preserve its extrusion profile and structural resolution during visible light-assisted 3D bioprinting. Therefore, in one embodiment, a temperature interval between 22° C. and 26° C. is selected to maintain optimal ink viscosity and ensure stable filament extrusion. To achieve this, the printing syringe is equipped with an integrated thermal regulation element, such as a flexible resistive heating jacket or a thermoelectric module, which is positioned in direct contact with the outer surface of the syringe barrel. The heating or cooling element is operatively connected to a digital temperature controller configured to maintain the syringe temperature within the desired range. The controller receives real-time input from a temperature sensor embedded near the nozzle or along the syringe wall, and continuously adjusts the thermal input to compensate for ambient fluctuations or extrusion-induced heat loss.

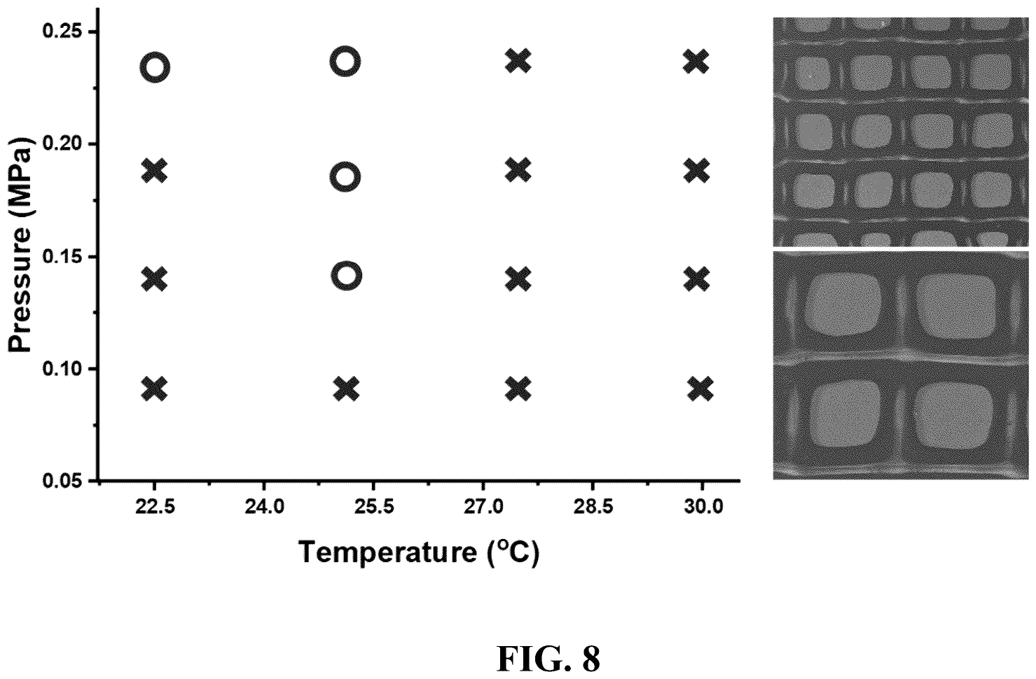

FIG. 8 illustrates the influence of printing parameters on scaffold formation, as applied in Example 4. The left panel presents a parameter matrix assessing the effect of nozzle gauge, extrusion pressure, and printing speed on scaffold fidelity. Combinations of these parameters were evaluated for their ability to yield uniform, continuous, and well-defined structures. The right panel shows representative images of printed scaffolds obtained under optimized conditions.

The test results indicate that only a narrow range of conditions (e.g., being about 25° C. with an extrusion pressure of 0.2+0.4 MPa) yields scaffolds with consistently uniform pore architecture and minimal distortion. In contrast, deviations from these parameters resulted in irregular filament deposition, non-uniform pore spacing, or loss of structural definition. The visual evidence on the right supports these findings: scaffolds printed under optimized conditions exhibit regular square lattice geometry with sharp edges and consistent line widths, demonstrating excellent print fidelity. These results confirm the importance of synergistic parameter tuning in achieving reproducible scaffold morphologies.

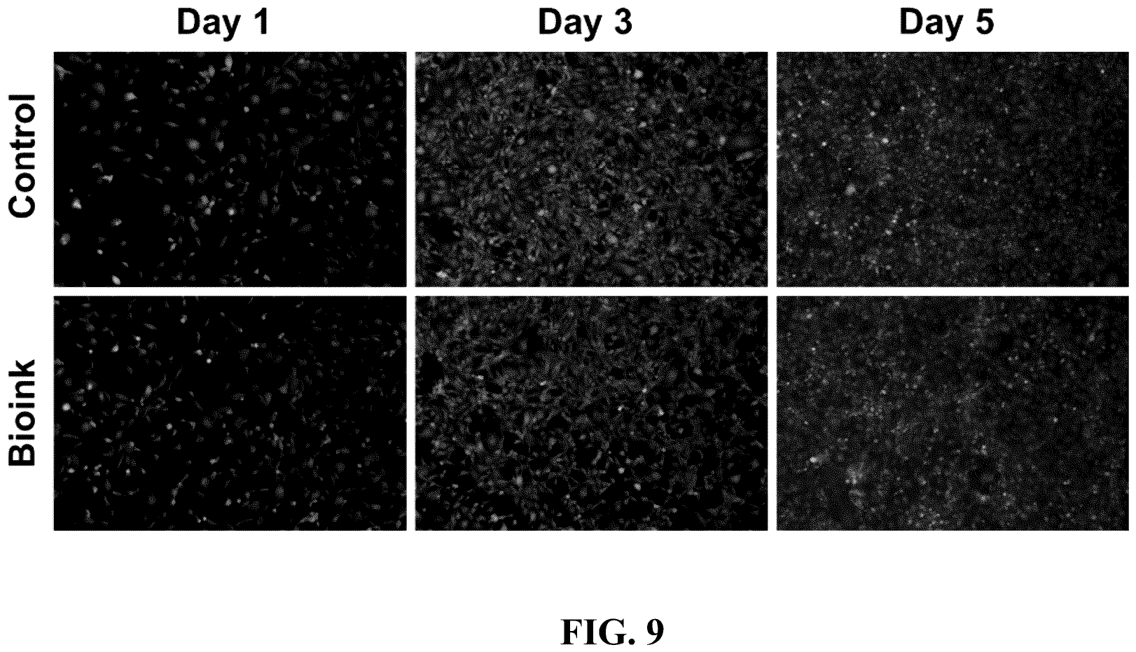

FIG. 9 shows fluorescence images of the biocompatibility of the printed scaffolds described in Example 4. The presence of predominantly viable cells indicates that the scaffold exhibits good biocompatibility.

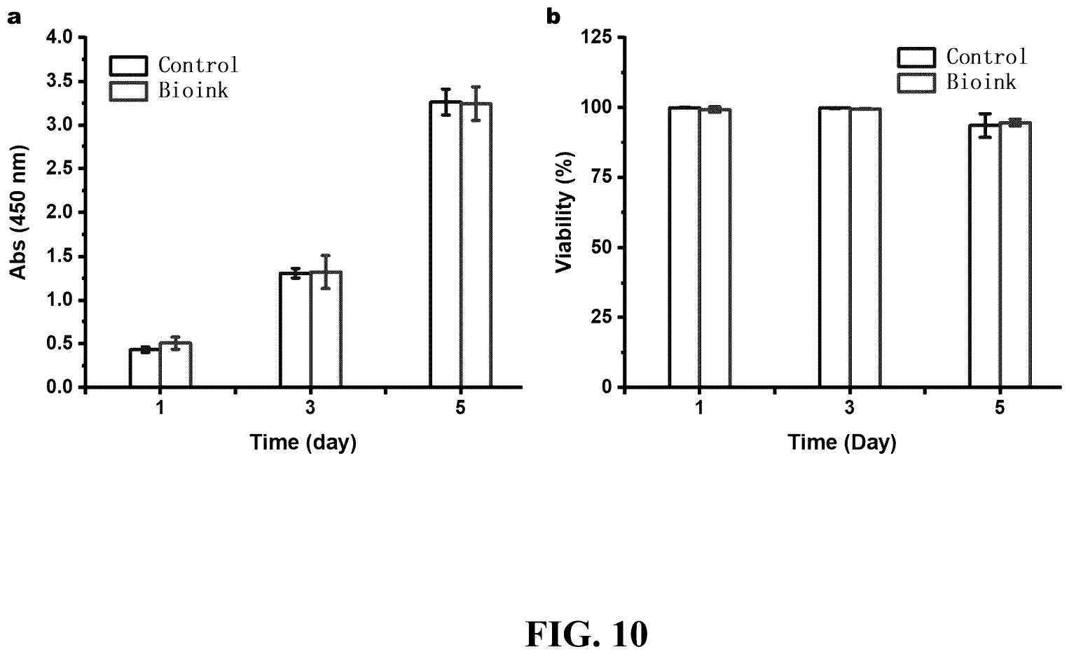

FIG. 10 presents the quantitative evaluation of cytotoxicity and cell proliferation for the scaffolds printed in Example 4. Part (a) of FIG. 10 shows the results of a cytotoxicity assay, indicating that the printed scaffolds exhibit minimal cytotoxic effects, with cell viability levels nearly identical to those of the blank control group. Part (b) of FIG. 10 shows cell proliferation measured over multiple time points. The results demonstrate a consistent increase in cell number, confirming that the scaffold supports cell growth over time. These findings verify that both the bio-ink formulation and the visible-light-assisted printing process are biocompatible and conducive to long-term cell culture.

### Example 5: Fabrication of a Cell-Laden 3D Printed Hydrogel Scaffold

Example 5 differs from Example 4 in two primary aspects. First, the bio-ink formulation is prepared using DEME culture medium instead of deionized water, thereby providing a cell-compatible environment during the printing process. Second, live NIH 3T3 fibroblasts are introduced into the ink formulation prior to printing, enabling the direct fabrication of cell-laden scaffolds. Example 5 is intended to demonstrate the feasibility of encapsulating viable cells within the hydrogel matrix and printing functional biological constructs under mild, visible light-induced crosslinking conditions.
1. Preparation of the Bio-Ink: One gram of methacrylated gelatin (degree of substitution: 60%) is added to 10 mL of DEME culture medium and stirred continuously at 50° C. until fully dissolved. After cooling to room temperature, 0.1 g of recombinant spider silk protein powder, as prepared in Example 2, is added to form Solution A. Separately, 60 mg of tris(bipyridine)ruthenium(II) chloride is dissolved in 4 mL of DEME medium and stored in the dark at 2-8° C. Likewise, 120 mg of ammonium persulfate is dissolved in another 4 mL of DEME medium under the same storage conditions. A volume of 0.5 mL from each of the two initiator solutions is added to Solution A and mixed thoroughly. The resulting mixture is passed through a 0.22 μm sterile filter. Then, 1 mL of NIH 3T3 mouse fibroblasts at a density of 10⁷ cells/mL is added to the filtered ink and gently mixed by pipetting to ensure homogeneous cell distribution.
2. 3D Printing of the Hydrogel Scaffold: The resulting cell-laden bio-ink is loaded into the syringe of a regenovo bioprinter. A 432 nm visible blue light source is directed onto the printing platform. The syringe temperature is set to 22.5° C. and the platform temperature is maintained at 10° C. After a 10-minute equilibration period to stabilize temperature gradients, printing is initiated under concurrent extrusion and light irradiation. The bio-ink is extruded through a 27G nozzle at a pressure of 0.20 MPa and a speed of 20 mm/s. Upon completion of the printing process, the structure is exposed to an additional 1 minute of visible light to complete crosslinking.

In this example, the resulting hydrogel scaffold has a length and width of 15 mm, a height of 6 mm, and an internal grid spacing of 0.54 mm.

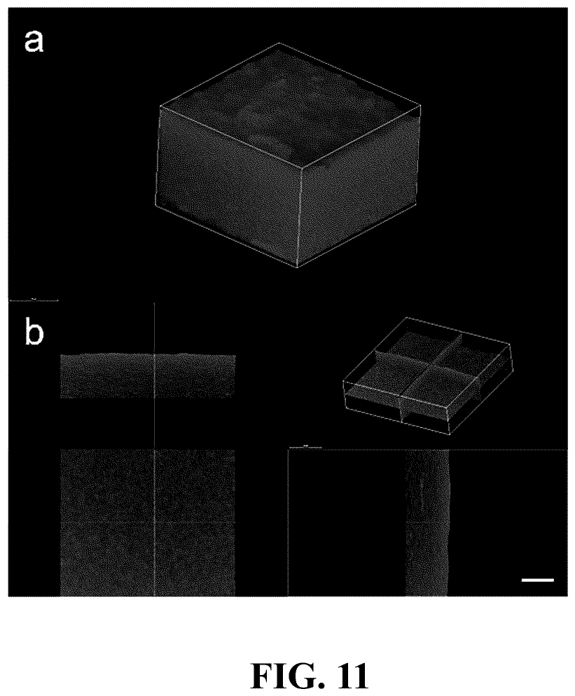

FIG. 11 shows confocal fluorescence microscopy images of the cell-laden scaffold printed in Example 5. The illustration confirm that cells are distributed throughout the hydrogel matrix and remain viable after the printing process. The result of FIG. 11 demonstrates that the scaffold provides a supportive environment for cell survival.

### Example 6: Validation of Printing Parameter Flexibility

This example follows the same procedure as described in Example 4, with the exception that the 3D printing parameters are modified. Specifically, the syringe temperature is set to 22.5° C., the extrusion speed is reduced to 15 mm/s, and the pneumatic pressure is increased to 0.25 MPa. The successful formation of structurally stable scaffolds under these alternative conditions demonstrates that the bio-ink formulation described in the present invention offers a degree of flexibility with respect to printing parameters. The ability to operate within a tunable window, rather than relying on narrow or fixed settings, indicates that the ink exhibits robust rheological behavior and predictable photocuring performance. This parameter tolerance is advantageous in practical bioprinting scenarios, where adjustments may be required to accommodate different printer models, nozzle configurations, or cellular load conditions.

In conclusion, the present invention provides a visible light-curable bio-ink based on recombinant spider silk protein, offering a streamlined and biocompatible solution for hydrogel-based 3D bioprinting. It includes a complete process covering protein expression, purification, ink formulation, and visible light-induced crosslinking. The bio-ink enables stable scaffold formation without the use of toxic crosslinkers, supporting cell viability and proliferation. Its formulation is simple, environmentally friendly, and compatible with standard printing equipment.

Additionally, the ink allows for flexible adjustment of printing parameters, enhancing its practical applicability in tissue engineering and biomedical applications.

### INDUSTRIAL APPLICABILITY

The bio-ink formulation provided in the present invention demonstrates enhanced printability by combining methacrylated gelatin (GelMA) with recombinant spider silk protein. This composition enables improved stability and precision during visible light-assisted 3D bioprinting, allowing the fabrication of complex and finely resolved biological structures. The use of recombinant spider silk protein also contributes to superior biocompatibility, supporting cell adhesion, proliferation, and differentiation, which makes the ink particularly suitable for biomedical applications such as tissue engineering and regenerative medicine.

The ink can be cured under visible light instead of ultraviolet light, significantly reducing potential cellular damage during the printing process. This mild curing condition is essential for maintaining cell viability and function within printed hydrogel scaffolds. Moreover, the ink formulation is free of toxic crosslinkers and complex chemical additives, making it environmentally friendly and well-suited for both laboratory and clinical use.

Due to its combined advantages in printability, cytocompatibility, and safety, the bio-ink has broad application potential. It may be employed in a variety of 3D bioprinting scenarios including, but not limited to, the fabrication of tissue scaffolds, disease models, and high-throughput drug screening platforms.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A photocrosslinkable bioink composition for three-dimensional printing, comprising:
an aqueous solution of a recombinant spider silk protein comprising the NT2RepCT-MaSp2 sequence;
a polymer comprising pendant groups reactive under visible light-initiated radical polymerization; and
a photoinitiator system configured to generate free radicals upon exposure to visible light,
wherein the polymer is selected from the group consisting of methacrylated gelatin, methacrylated hyaluronic acid, methacrylated chitosan, polyethylene glycol diacrylate, and combinations thereof,
wherein the photoinitiator system is selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate,
wherein the photocrosslinkable bioink composition is curable under visible light to form a biocompatible hydrogel structure suitable for cell culture or implantation.

2. The photocrosslinkable bioink composition of claim 1, wherein the photoinitiator system comprises tris(bipyridine)ruthenium(II) chloride and ammonium persulfate at a concentration configured to initiate crosslinking upon exposure to light in the range of 450 to 520 nanometers.

3. The photocrosslinkable bioink composition of claim 1, wherein the recombinant spider silk protein comprises one or more terminal groups functionalized with methacrylate or acrylate moieties to facilitate covalent incorporation into the photocrosslinked network.

4. The photocrosslinkable bioink composition of claim 1, wherein the photocrosslinkable bioink composition further comprises one or more fibroblasts suspended in the aqueous phase, and wherein the photocrosslinking conditions are cytocompatible such that cell viability of the one or more fibroblasts is retained following visible light exposure.

5. The photocrosslinkable bioink composition of claim 1, wherein the polymer comprises methacrylated gelatin and the resulting cured hydrogel exhibits an elastic modulus between 5 and 50 kilopascals, suitable for soft tissue applications.

6. A method of fabricating a biocompatible three-dimensional hydrogel construct, comprising:
providing a bioink composition comprising:
a recombinant spider silk protein comprising the NT2RepCT-MaSp2 sequence;
a visible light-reactive polymer selected from the group consisting of methacrylated gelatin, methacrylated hyaluronic acid, methacrylated chitosan, polyethylene glycol diacrylate, and combinations thereof; and
a photoinitiator selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate;
dispensing the bioink composition into a defined three-dimensional pattern using a printing device; and
exposing the patterned bioink to visible light in the range of 405 to 520 nanometers to induce photocrosslinking and form a structurally stable hydrogel, wherein a resulting printed construct is configured to support cell adhesion, proliferation, or tissue integration.

7. The method of claim 6, wherein the visible light exposure is provided by a digital light projector or laser emitting light at 432 nanometers.

8. The method of claim 6, wherein the printed construct is configured as a wound healing scaffold comprising a porous hydrogel network infused with the spider silk protein, and wherein the scaffold supports epithelial cell migration across the wound bed.

9. The method of claim 6, wherein the printed construct comprises a tissue engineering scaffold having a three-dimensional lattice structure configured to promote attachment and proliferation of fibroblasts, chondrocytes, or stem cells.

10. The method of claim 6, wherein the bioink composition further comprises one or more fibroblasts suspended in the aqueous phase, and wherein the photocrosslinking conditions are cytocompatible such that cell viability of the one or more fibroblasts is retained following the exposing the patterned bioink to the visible light.

11. The method of claim 6, wherein the dispensing the bioink composition into the defined three-dimensional pattern is performed using the printing device with a temperature maintained between approximately 22.5° C. and 25° C.

12. A three-dimensional (3D) printing bioink composition, comprising:
a spider silk protein;
a photocrosslinkable polymer comprising a pendant group reactive under visible light;
a visible light photoinitiator; and
a biocompatible aqueous carrier.

13. The 3D printing bioink composition of claim 12, wherein the spider silk protein is a recombinant protein comprising repeat units selected from glycine-alanine motifs or poly-alanine blocks.

14. The 3D printing bioink composition of claim 12, wherein the photocrosslinkable polymer is selected from the group consisting of methacrylated gelatin, methacrylated hyaluronic acid, methacrylated chitosan, polyethylene glycol diacrylate, norbornene-functionalized polyethylene glycol, and combinations thereof.

15. The 3D printing bioink composition of claim 12, wherein the visible light photoinitiator is selected from the group consisting of tris(bipyridine)ruthenium(II) chloride with ammonium persulfate, eosin Y with triethanolamine, riboflavin with ammonium persulfate, and lithium phenyl-2,4,6-trimethylbenzoylphosphinate.

16. A method of fabricating a biocompatible three-dimensional structure, comprising:
providing the composition of claim 12;
extruding the composition into a defined geometry; and
exposing the extruded material to visible light to form a crosslinked hydrogel structure.


SEQUENCE LISTING (6 sequences)

SEQ ID NO: 1 - DNA, 32 bp, synthetic construct
  source 1..32: mol_type = other DNA
  misc_feature 1..32: note = PCR_primers_fwd_seq
aggaggatatggtcaaggtgctggcggtaatg

SEQ ID NO: 2 - DNA, 32 bp, synthetic construct
  source 1..32: mol_type = other DNA
  misc_feature 1..32: note = PCR_primers_res_seq
cttgaccatatcctccttgcccttgcccagca

SEQ ID NO: 3 - DNA, 42 bp, synthetic construct
  source 1..42: mol_type = other DNA
  misc_feature 1..42: note = PCR_primers_fwd_seq
tagaggaccaggtcaaggaggatatggaccaggtgccggaag

SEQ ID NO: 4 - DNA, 40 bp, synthetic construct
  source 1..40: mol_type = other DNA
  misc_feature 1..40: note = PCR_primers_rev_seq
cttgacctggtcctctataaccaccttgaccaccttgttg

SEQ ID NO: 5 - DNA, 32 bp, synthetic construct
  source 1..32: mol_type = other DNA
  misc_feature 1..32: note = PCR_primers_fwd_seq
aggaggatacggtcaaggtgctccaggcgtta

SEQ ID NO: 6 - DNA, 36 bp, synthetic construct
  source 1..36: mol_type = other DNA
  misc_feature 1..36: note = PCR_primers_rev_seq
cttgaccgtatcctcctcttccttgtccaccttgac